(19) United States
(12) Patent Application Publication
(10) Pub. No.: US 2013/0281410 A1
(43) Pub. Date: Oct. 24, 2013

(54) METHODS FOR THE TREATMENT OF PSYCHIATRIC DISORDERS

(71) Applicant: Perry F. Renshaw, Salt Lake City, UT (US)

(72) Inventor: Perry F. Renshaw, Salt Lake City, UT (US)

(73) Assignees: University of Utah Research Foundation, Salt Lake City, UT (US); The McLean Hospital Corporation, Belmont, MA (US)

(21) Appl. No.: 13/697,674

(22) PCT Filed: May 13, 2011
(86) PCT No.: PCT/US11/36405

§ 371 Date: May 29, 2013

Related U.S. Application Data
(60) Provisional application No. 61/334,373, filed on May 13, 2010.

(51) Int. Cl. A61K 31/198; A61K 45/06

(52) U.S. Cl. CPC A61K 31/198; A61K 45/06
USPC 514/120; 514/565; 514/392; 514/275

(57) ABSTRACT

The invention provides methods for the treatment of major depressive disorder in male subjects and methods for the treatment of psychiatric disorders at high altitudes by administering one or more creatine-containing compounds.

### CROSS-REFERENCE TO RELATED APPLICATIONS

This application claims benefit of U.S. Provisional Application No. 61/334,373, filed May 13, 2010, which is hereby incorporated by reference.

### BACKGROUND OF THE INVENTION

In general, the invention relates to the treatment of psychiatric disorders.

Major depressive disorder (MDD) is a prevalent disorder that is often chronic and associated with frequent relapses and long duration of episodes. This disorder includes psychosocial and physical impairment and a high suicide rate among those affected. A lifetime prevalence of approximately 17% has been widely reported, and the likelihood of recurrence is more than 50% (Angst. J. Clin. Psychiatry 60 Suppl. 6:5-9, 1999). The neurological mechanisms underlying MDD are poorly understood, with a concomitant lack in suitable pharmacological therapies for the treatment of this disorder.

Antidepressants are commonly used to treat MDD and other psychiatric disorders. The most widely used antidepressants are selective serotonin reuptake inhibitors (SSRIs). SSRIs have generally been regarded as safe and effective in MDD subjects. However, one of the major challenges frequently met in routine clinical practice is that there are few proven treatment options for MDD patients who are considered “treatment-resistant” to conventional antidepressant therapy with an adequate dose and duration. Furthermore, the mechanisms and the biological correlates of treatment resistance in MDD patients have not been well-characterized. Additional therapies for MDD and other psychiatric disorders are presently desired.

### SUMMARY OF THE INVENTION

The invention provides methods for treating MDD in male subjects by administering to the male a therapeutically-effective amount of a creatine-containing compound. In different embodiments of these methods, the male may be a child, an adolescent, or an adult aged 60 years or older.

The invention also provides methods for treating a psychiatric disorder (e.g., MDD) in a subject living at high altitude by administering to the subject a therapeutically-effective amount of a creatine-containing compound. In different embodiments of the invention, the psychiatric disorder may be MDD, bipolar disorder, acute stress disorder, adjustment disorder, agoraphobia, antisocial personality disorder, anxiety disorder, avoidant personality disorder, body dysmorphic disorder, borderline personality disorder, brief psychotic disorder, conversion disorder, cyclothymic disorder, delusional disorder, dependent personality disorder, depersonalization disorder, dissociative disorder, depressive disorder, dysthymic disorder, gender identity disorder, hypochondriasis, impulse control disorder, intermittent explosive disorder, kleptomania, narcissistic personality disorder, obsessive compulsive disorder, paranoid personality disorder, posttraumatic stress disorder, psychotic disorders, schizophrenia, shared psychotic disorder, specific phobia, or attention deficit hyperactivity disorder. In different embodiments of these methods, the subject lives at an elevation of greater than 500 feet above sea level or at an elevation of greater than 1000 feet above sea level. In additional embodiments of these methods, the subject may be a child, an adolescent, or an adult aged 60 years or older.

In all the above methods of the invention, the creatine-containing compound may contain a creatine salt or a creatine analog. In all the above methods of the invention, the creatine analog may be selected from the group of: cyclocreatine, homocyclocreatine, 3-guanidinopropionic acid, and 1-carboxymethyl-2-iminohexahydropyrimidine. In additional embodiments of all the above methods, the creatine-containing compound may be administered orally (e.g., one or more times a day). For example, the creatine-containing compound may be administered orally four times a day.

In additional embodiments of all the above methods, the creatine-containing compound may be administered in a dose of 50 mg per day to 20,000 mg per day. Further, the administering may require administration of more than one dose.

In additional embodiments of the invention, the method further requires administering to the male or subject a brain phospholipid or a brain phospholipid precursor, e.g., a fatty acid, glycerol, a lipid, or sphingosine. In additional embodiments of the invention, the method further includes administering to the subject one or more additional agents, e.g., a pyrimidine, an antidepressant, an anticonvulsant, an antianxiety, an antimanic, an antipsychotic, an antiobsessional, a sedative-hypnotic, a stimulant, an anti-hypertensive medication, or a selective serotonin reuptake inhibitor (SSRI).

The invention also features a creatine-containing compound for use in any of the methods described herein or for use in the manufacture of a medicament for use in any of the methods described herein.

By “major depressive disorder” or “MDD” is meant a clinical course that is characterized by one or more major depressive episodes in an individual without a history of manic, mixed, or hypomanic episodes. The diagnosis of MDD is not made if: manic, mixed, or hypomanic episodes develop during the course of depression; if the depression is due to the direct physiological effects of a substance; if the depression is due to the direct physiological effects of a general medical condition; if the depression is due to a bereavement or other significant loss (“reactive depression”), or if the episodes are better accounted for by schizoaffective disorder and are not superimposed on schizophrenia, schizophreniform disorder, delusional disorder, or psychotic disorder. If manic, mixed, or hypomanic episodes develop, then the diagnosis is changed to bipolar disorder. Depression may be associated with chronic general medical conditions (e.g., diabetes, myocardial infarction, carcinoma, and stroke). Generally, MDD is more severe than dysthymia.

The essential feature of major depressive episode is a period of at least two weeks during which there is either depressed mood or loss of interest or pleasure in nearly all activities. In children and adolescents, the mood may be irritable rather than sad. The episode may be a single episode or may be recurrent. The individual also experiences at least four additional symptoms drawn from a list that includes changes in appetite or weight, sleep, and psychomotor activity; decreased energy; feelings of worthlessness or guilt; difficulty thinking, concentrating, or making decisions; or recurrent thoughts of death or suicidal ideation, plans, or attempts. Each symptom must be newly present or must have clearly worsened compared with the person's pre-episode status. The symptoms must persist for most of the day, nearly every day, for at least two consecutive weeks, and the episode must be accompanied by clinically significant distress or impairment in social, occupational (or academic), or other important areas of functioning. (See, Diagnostic and Statistical Manual of Mental Disorders (DSM IV), American Psychiatric Press, 4^{th} Edition, 1994).

By “treating” is meant the medical management of a patient with the intent that amelioration or reduction in one or more symptoms (e.g., two, three, or four) of the disease, pathological condition, or disorder will result. This term includes active treatment, that is, treatment directed specifically toward improvement of a disease, pathological condition, or disorder, and also includes causal treatment, that is, treatment directed toward removal of the cause of the disease, pathological condition, or disorder. In addition, this term includes palliative treatment, that is, treatment designed for the relief of symptoms rather than the curing of the disease, pathological condition, or disorder; and supportive treatment, that is, treatment employed to supplement another specific therapy directed toward the improvement of the disease, pathological condition, or disorder. The term “treating” also includes symptomatic treatment, that is, treatment directed toward constitutional symptoms of the disease, pathological condition, or disorder.

By “preventing” is meant treatment that is directed to prevent or delay the onset or development of one or more (e.g., two, three, four, or five) symptoms of the disease, pathological condition, or disorder.

By “therapeutically-effective amount” is meant an amount of a creatine-containing compound sufficient to produce a healing, curative, prophylactic, stabilizing, or ameliorative effect in the treatment of a psychiatric disorder (e.g., MDD).

By “more effective” is meant that a treatment exhibits greater efficacy, or is less toxic, safer, more convenient, or less expensive than another treatment with which it is being compared. More effective may also mean the ability to decrease the severity or alleviate one or more symptoms of a psychiatric disease (e.g., MDD) in a subject that is resistant to other therapies. Efficacy may be measured by a skilled practitioner using any standard method that is appropriate for a given indication.

By “creatine-containing compound” is meant any compound that includes as a component, creatine. A creatine-containing compound may contain a creatine salt or a creatine analog. Non-limiting examples of creatine analogs include: cyclocreatine, homocyclocreatine, 3-guanidinopropionic acid, and 1-carboxymethyl-2-iminohexahydropyrimidine. Another non-limiting example of a creatine-containing compound is creatine monohydrate.

By “phospholipid” is meant a lipid containing phosphorus, e.g., phosphatidic acids (e.g., lecithin), phosphoglycerides, sphingomyelin, and plasmalogens. By “phospholipid precursor” is meant a substance that is built into a phospholipid during synthesis of the phospholipid, e.g., fatty acids, glycerol, or sphingosine.

By “child or adolescent” is meant an individual who has not attained complete growth and maturity. Generally, a child or adolescent is under twenty-one years of age.

By “adult” is meant an individual who is 21 years of age or older.

By “older adult” is meant an individual who is in the later stage of life.

Generally, an older adult is over sixty years of age.

By “high altitude” is meant an elevation that is greater than 200 feet above sea level. For example, high altitude may be an elevation: greater than 300 feet above sea level, greater than 400 feet above sea level, greater than 500 feet above sea level, greater than 600 feet above sea level, greater than 700 feet above sea level, greater than 800 feet above sea level, greater than 900 feet above sea level, greater than 1000 feet above sea level, greater than 1,500 feet above sea level, greater than 2,000 feet above sea level, greater than 2,500 feet above sea level, greater than 3,000 feet above sea level, greater than 3,500 feet above sea level, greater than 4,000 feet above sea level, greater than 4,500 feet above sea level, or greater than 5,000 feet above sea level.

By “subject living at a high altitude” is meant a person who spends greater than 30% (e.g., greater than 40%, 50%, 60%, 70%, 80%, 90%, or even 100%) of a 24-hour period at a high altitude (as defined above) over an extended period of time (e.g., greater than 3 days, greater than 1 week, greater than 1 month, and greater than 1 year). For example, a subject living at a high altitude may have their permanent residence or primary residence at a location that is at high altitude. In another example, a subject living at a high altitude may work in a location (e.g., in an office) or an environment (e.g., an airplane) that is at high altitude. In another example, a subject may be visiting or temporarily located (e.g., greater than one week) at a high altitude.

By “psychiatric disorder” is a psychological or behavioral pattern that occurs in an individual and is thought to cause distress or disability that is not expected as part of normal development or culture. Examples of psychiatric disorders are described in the Diagnostic and Statistical Manual of Mental Disorders, 4th. Edition (DSM-IV). DSM-IV is published by the American Psychiatric Association and describes mental health disorders for both children and adults. DSM-IV also provides information regarding the diagnosis and symptoms of psychiatric disorders.

Non-limiting examples of psychiatric disorders include: major depressive disorder (MDD), bipolar disorder, acute stress disorder, adjustment disorder, agoraphobia, antisocial personality disorder, anxiety disorder, avoidant personality disorder, body dysmorphic disorder, borderline personality disorder, brief psychotic disorder, conversion disorder, cyclothymic disorder, delusional disorder, dependent personality disorder, depersonalization disorder, dissociative disorder, depressive disorder, dysthymic disorder, gender identity disorder, hypochondriasis, impulse control disorder, intermittent explosive disorder, kleptomania, narcissistic personality disorder, obsessive compulsive disorder, paranoid personality disorder, posttraumatic stress disorder, psychotic disorders, schizophrenia, shared psychotic disorder, specific phobia, and attention deficit hyperactivity disorder.

By “antidepressant” is meant a psychiatric medication used to alleviate mood disorders, such as major depression disorder and dysthymic disorder. Drugs including the monoamine oxidase inhibitors (e.g., isocarboxazid, moclobemide, phenelzine sulfate, selegiline, and tranylcypromine sulfate), tricyclic antidepressants (amitriptyline, amitriptyline hydrochloride, amitriptylinoxide, butriptyline, clomipramine, demexiptiline, desipramine, dibenzepin, dimetacrine, dosulepin/dothiepin, doxepin, imipramine, imipraminoxide, lofepramine, melitracen, metapramine, nitroxazepine, nortriptyline, noxiptilinc, pipofezine, propizepine, protriptyline hyrdochloride, and quinupramine), tetracyclic antidepressants (amoxapine, loxapine, maprotiline, mazindol, mianserin, mirtazapine, and setiptiline), selective serotonin reuptake inhibitors (SSRIs; see below), and serotonin-norepinephrine reuptake inhibitors (SNRIs; e.g., desvenlafaxine, duloxetine, milnacipram, and venlafaxine hydrochloride) are most commonly associated with the term. Additional examples of antidepressants include bupropion hydrochloride, imipramine hydrochloride, lamotrigine, trazodone hydrochloride, and trimipramine maleate.

Most typical antidepressants have a delayed onset of action (2-6 weeks) and are usually administered for months to years. Despite their name, antidepressants are often used to treat other conditions, such as anxiety disorders, obsessive compulsive disorder, eating disorders, chronic pain, and some hormone-mediated disorders such as dysmenorrhea.

By “selective serotonin reuptake inhibitor” or “SSRI” is meant an antidepressant that works by preventing the reuptake of serotonin by the presynaptic neuron, thus maintaining higher levels of 5-HT in the synapse. Non-limiting examples of SSRIs include: citalopram, dapoxetine, escitalopram, fluoxetine, fluvoxamine, indalpine, paroxetine, and sertraline.

By “anticonvulsant medication” is meant an agent that is used in the treatment of epileptic seizures. The goal of an anticonvulsant is to suppress the rapid and excessive firing of neurons that start a seizure. Anticonvulsants have also proved effective in treating many kinds of psychiatric disorders (e.g., dysfunctional anxiety). Non-limiting examples of anticonvulsant drugs include: amobarbital, amobarbital sodium, paraldehyde, stiripentol, phenobarbital, phenobarbital sodium, methylphenobarbital, metharbital, barbexaclone, chlordiazepoxide, chlordiazepoxide hydrochloride, clobazam, clonazepam, clorazepate dipotassium, diazepam, divalproex sodium, midazolam, lorazepam, potassium bromide, felbamate, carbamazepine, oxcarbazepine, eslicarbazepine acetate, valproic acid, sodium valproate, divalproex sodium, vigabatrin, progabide, tiagabine, topiramate, gabapentin, pregabalin, ethotoin, phenyloin, phenyloin sodium, mephenyloin, fosphenyloin, paramethadione, trimethadione, ethadione, beclamide, magnesium sulfate, phensuximide, primidone, brivaracetam, levetiracetam, seletracetam, ethosuximide, phesuximide, mesuximide, mephobarbital, acetazolamide, sultiame, secobarbital sodium, methazolamide, methsuximide, zonisamide, lamotrigine, pentobarbital sodium, pheneturide, phenacemide, valpromide, and valnoctamide.

By “anti-anxiety medication” is meant an agent that reduces anxiety or an anxiety-related psychiatric related disorder in a subject. Non-limiting examples of anti-anxiety medications include alprazolam, buspirone hydrochloride, chlordiazepoxide, chlordiazepoxi de hydrochloride, clorazepate dipotassium, clonazepam, desipramine hydrochloride, diazepam, halazepam, hydroxyzine hydrochloride, hydroxyzine pamoate, lorazepam, meprobamate, oxazepam, prazepam, prochlorperazine maleate, prochlorperazine, prochlorperazine edisylate, and trimipramine maleate.

By “anti-manic medication” is meant an agent that reduces or decreases the severity of manic episodes in a subject. Non-limiting examples of anti-manic medications include carbamazepine, divalproex sodium, gabapentin, lamotrigine, topimarate, lithium carbonate, and lithium citrate.

By “antiobsessional medication” is meant an agent that reduces or ameliorates one or more symptoms of an obsessional disorder. Obsessional disorders are characterized by intrusive thoughts that produce anxiety, by repetitive behaviors aimed at reducing anxiety, or by a combination of such thoughts and behaviors. Non-limiting examples of antiobsessional medications include fluvoxamine and clomipramine hydrochloride.

By “anti-psychotic medication” is meant an agent that is primarily used to manage psychosis (e.g., delusions and hallucinations). Non-limiting examples of anti-psychotic medications include acetophenazine maleate, amisulpride, aripiprazole, asenapine, cannabidiol, clopenthixol, droperidol, chlorpromazine hydrochloride, chlorprothixene, chlorprothixene hydrochloride, clozapine, fluphenazine decanoate, fluphenazine enathrate, fluphenazine hydrochloride, flupenthixol, haloperidol decanoatc, haloperidol, haloperidol lactate, iloperidone, lithium carbonate, lithium citrate, levomepromazine, loxapine hydrochloride, loxapine succinate, mesoridazine besylate, molindone hydrochloride, olanzapine, paliperidone, perphenazine, periciazine, pimozide, prochlorperazine maleate, prochlorperazine, prochlorperazine edisylate, promethazine, promazine hydrochloride, quetiapine, risperidone, sertindole, thioridazine, tetrabenazine, thioridazine hydrochloride, thiothixene, thiothixene hydrochloride, triflupromazine, trifluoperzine hydrochloride, zuclopenthixol, ziprasidone, and zotepine.

By “sedative-hypnotic medication” is meant an agent used to reduce tension and induce calm or sleep. Non-limiting examples of sedative-hypnotic agents include amobarbital, amobarbital sodium, aprobarbital, butabarbital, chloral hydrate, chlordiazepoxide, chlordiazepoxide hydrochloride, clorazepate dipotassium, ethchlorvynol, flurazepam hydrochloride, glutethimide, hydroxyzine hydrochloride, pentobarbital sodium, pentobarbital, secobarbital, secobarbital sodium, phenobarbitol, phenobarbital sodium, benzodiazepines, clonazepam, diazepam, estazolam, flunitrazepam, lorazepam, methotrimeprazine hydrochloride, midazolam hydrochloride, nitrazepam, oxazepam, triazolam, temazepam, alprazolam, eszopiclone, zaleplon, zolpidem, zolpidem tartrate, zopiclone, diphenhydramine, dimenhydramine, dimenhydrinate, doxylamine, phenergan, promethazine, and quazepam.

By “stimulant” is meant an agent that induces temporary improvements in either mental or physical function or both. Examples of these kinds of effects may include enhanced alertness, wakefulness, and locomotion. Non-limiting examples of stimulants include caffeine, nicotine, amphetamine, dextroamphetamine sulfate, methamphetamine, methamphetamine hydrochloride, methylenedioxymethamphetamine, methylphenidate, methylphenidate hydrochloride, bupropion, atomoxetine, pemoline, reboxetine, modafinil, ampalex, CX717, carphedon, and yohimbine.

By “anti-hypertensive medication” is meant an agent that reduces blood pressure in a subject. Non-limiting examples of anti-hypertensive medication include: clonidine, bumetanide, ethacrynic acid, furosemide, torsemide, epitizide, hydrochlorothiazide, chlorothiazide, bendroflumethiazide, indapamide, chlorthalidone, metolazone, amiloride, triamterene, spironolactone, atenolol, metoprolol, nadolol, oxprenolol, pindolol, propranolol, timolol, doxazosin, phentolamine, indoramin, phenoxybenzamine, prazosin, terazosin, tolazoline, bucindolol, carvedilol, labetalol, clonidine, methyldopa, guanfacine, amlodipine, felodipine, isradipine, lercanidipine, nicardipine, nifedipine, nimodipine, nitrendipine, diltiazem, verapamil, captopril, enalapril, fosinopril, lisinopril, perindopril, quinapril, ramipril, trandolapril, benazepril, candesartan, eprosartan, irbesartan, losartan, olmesartan, telmisartan, valsartan, eplerenone, spironolactone, sodium nitroprusside, hydralazine, clonidine, methyldopa, and moxonidine.

By “sustained release” or “controlled release” is meant that the therapeutically active component is released from the formulation at a controlled rate such that therapeutically beneficial blood levels (but below toxic levels) of the component are maintained over an extended period of time ranging from e.g., about 12 to about 24 hours, thus, providing, for example, a 12 hour or a 24 hour dosage form.

The term “pharmaceutically acceptable salt” represents those salts which are, within the scope of sound medical judgement, suitable for use in contact with the tissues of humans and lower animals without undue toxicity, irritation, allergic response and the like, and are commensurate with a reasonable benefit/risk ratio. Pharmaceutically acceptable salts are well known in the art. The salts can be prepared in situ during the final isolation and purification of the compounds of the invention, or separately by reacting the free base function with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, isethionate, lactobionate, lactate, laurate, lauryl sulfate, malate, maleate, malonate, mesylate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethyl ammonium, methylamine, dimethylamine, trimethylamine, triethylamine, ethylamine, and the like.

The creatine-containing compounds utilized herein are relatively non-toxic, pharmocokinetically understood, and known to be well tolerated by mammals. The present invention, therefore, provides treatments for MDD in males and psychiatric disorders in subjects at high altitudes that are likely to have few adverse effects.

### BRIEF DESCRIPTION OF THE DRAWINGS

FIGS. 1A and 1B show clinical assessment data of male subjects having MDD upon treatment with placebo (grey lines) or creatine monohydrate (5 g/day, black lines) for 0 to 8 weeks. Clinical assessment was performed using the Hamilton Depressing Rating Scale (FIG. 1A) or the Beck Depression Inventory scale (FIG. 1B).

FIG. 2 shows clinical assessment data of three adolescent subjects having MDD upon treatment with creatine monohydrate (5 g/day) in Salt Lake City (altitude of 4,300 feet) for 0 to 10 weeks, after failing a trial of selective serotonin reuptake inhibitor (SSRI). Clinical assessment was performed using the Children's Depression Rating Scale (CDRS).

### DETAILED DESCRIPTION

The invention provides methods for treating MDD in males and psychiatric disorders in subjects living at high altitude by administering a creatine-containing compound. The creatine-containing compound may be co-administered or co-formulated with one or more other compounds that are effective for the treatment of a psychiatric disorder (e.g., a pyrimidine, an antidepressant, an anticonvulsant, an antianxiety, an antimanic, an antiobsessional, an antipsychotic, a sedative-hypnotic, a stimulant, an anti-hypertensive medication, and a selective serotonin reuptake inhibitor, specific examples of which are provided herein).

### Major Depressive Disorder

The invention provides methods for the treatment of major depressive disorder (MDD) in males. A subject may be diagnosed as having MDD by a skilled physician based on the criteria set forth, for example, in the DSM-IV. A subject may also be identified as being at risk for developing a psychiatric disorder based on familial analysis.

A subject having MDD may have one or more symptoms (e.g., two, three, four, or five) of MDD selected from: depressed mood, loss of interest or pleasure, significant weight loss when not dieting or weight gain (e.g., a change of more than 5% of body weight in a month) or decrease in appetite, insomnia or hypersomnia, psychomotor agitation or retardation, fatigue or loss of energy, feelings of worthlessness or excessive or inappropriate guilt, diminished ability to think or concentrate, and recurrent thoughts of death. The symptoms of MDD cause clinically significant distress or impairment in social, occupational, or other important areas of functioning.

Desirably, administration of a creatine-containing compound to a male subject having MDD provides for alleviation of one or more symptoms (e.g., two, three, four, or five) symptoms of MDD in a subject. The administration of a creatine-containing compound may provide for alleviation of one or more symptoms of MDD in a male subject for an extended period of time (e.g., at least one week, two weeks, three weeks, a month, two months, three months, four months, five months, six months, a year, two years, three years, five years, or ten years).

### Psychiatric Disorders

The invention provides methods for the treatment of psychiatric disorders in subjects living at high altitude. A subject may be diagnosed as having a psychiatric disorder by a skilled physician based on the criteria, for example, set forth in the DSM-IV. A subject may also be identified as being at risk for developing a psychiatric disorder based on familial analysis.

Non-limiting examples of psychiatric disorders that may be treated by the methods of the invention include: major depressive disorder, bipolar disorder, acute stress disorder, adjustment disorder, agoraphobia, antisocial personality disorder, anxiety disorder, avoidant personality disorder, body dysmorphic disorder, borderline personality disorder, brief psychotic disorder, conversion disorder, cyclothymic disorder, delusional disorder, dependent personality disorder, depersonalization disorder, dissociative disorder, depressive disorder, dysthymic disorder, gender identity disorder, hypochondriasis, impulse control disorder, intermittent explosive disorder, kleptomania, narcissistic personality disorder, obsessive compulsive disorder, paranoid personality disorder, posttraumatic stress disorder, psychotic disorders, schizophrenia, shared psychotic disorder, specific phobia, and attention deficit hyperactivity disorder.

The symptoms of the above-listed psychiatric disorders are well-known by skilled physicians and are specifically described in the DSM-IV. The administration of a creatine-containing compound may provide for alleviation of one or more symptoms (e.g., two, three, four, or five) of a psychiatric disorder in a subject living at high altitude. The administration of a creatine-containing compound may provide for allevation of one or more symptoms of a psychiatric disorder in a subject living at high altitude for an extended period of time (e.g., at least one week, two weeks, three weeks, a month, two months, three months, four months, five months, six months, a year, two years, three years, five years, or ten years).

### Creatine-Containing Compounds

Creatine-containing compounds provide useful therapies because these compounds, by virtue of increasing brain phosphocreatine levels, can raise the levels of ATP. A creatine-containing compound may be a creatine salt or a creatine-analogue. Non-limiting examples of creatine salts include cyclocreatine, homocyclocreatine, 3-guanidinopropionic acid, and 1-carboxylmethyl-2-iminohexahydropyrimidine. Another example of a creatine-containing compound is creatine monohydrate.

Creatine has been established to be a safe natural product and has been introduced as a dietary supplement. Reported side effects of creatine administration include increased body mass, muscle cramping, diarrhea, gastrointestinal pain, and renal dysfunction. Despite a few recent anecdotal reports of serious renal effects, creatine supplementation has, in general, not been associated with major health risks.

### Administration

Conventional pharmaceutical practice is employed to provide suitable formulations or compositions for administration to patients. Oral administration is preferred, but any other appropriate route of administration may be employed, for example, parenteral, intravenous, subcutaneous, intramuscular, intracranial, intraorbital, ophthalmic, intraventricular, intracapsular, intraspinal, intracisternal, intraperitoneal, intranasal, or aerosol administration. Therapeutic formulations may be in the form of liquid solutions or suspensions (as, for example, for intravenous administration); for oral administration, formulations may be in the form of liquids, tablets, capsules, or as a food supplement (e.g., a solid, such as a nutritional bar, or a liquid, such as a shake); and for intranasal formulations, in the form of powders, nasal drops, or aerosols.

Methods well known in the art for making formulations are described, for example, in Remington: The Science and Practice of Pharmacy (21st ed., 2005). Formulations for parenteral administration may, for example, contain excipients, sterile water, saline, polyalkylene glycols such as polyethylene glycol, oils of vegetable origin, or hydrogenated naphthalenes.

If desired, slow release or extended release delivery systems may be utilized. Biocompatible, biodegradable lactide polymer, lactide/glycolide copolymer, or polyoxyethylene-polyoxypropylene copolymers may be used to control the release of the compounds. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

Preferably, the compounds of the invention (e.g., one or more creatine-containing compound), are administered at a dosage of 10 mg to 20,000 mg a day (e.g., between 50 mg to 20,000 mg a day, 100 mg to 20,000 mg a day, 200 mg to 20,000 mg a day, 500 mg to 20,000 mg a day, 1,000 mg to 20,000 mg a day, 5,000 mg to 20,000 mg a day, 10,000 mg to 20,000 mg a day, 10 mg to 10,000 mg a day, and 10 mg to 5,000 mg a day). The compounds of the invention may be administered one or more times (e.g., twice, three-times, or four-times) daily by oral administration. For example, a creatine-containing compound may be administered four times a day at a dose of 10 mg to 5,000 mg.

In general, the compounds of the invention (e.g., one or more creatine-containing compounds) are administered at a dosage appropriate to the effect to be achieved and are typically administered in unit dosage form. The exact dosage of the compound may be dependent, for example, upon the age and weight of the recipient, the route of administration, and the severity and nature of the symptoms to be treated. In general, the dosage selected should be sufficient to prevent, ameliorate, or treat MDD in a male subject or a psychiatric disorder in a subject living a high altitude, or one or more symptoms thereof, without producing significant toxic or undesirable side effects. As noted above, the preferred route of administration for most indications is oral. Creatine and creatine-containing compounds are known to be well tolerated at relatively high doses in humans.

### Combination Therapies

The creatine-containing compounds of the invention may be administered as a monotherapy, in combination with one or more (e.g., two, three, or four) additional creatine-containing compounds, or in combination with one or more (e.g., two, three, four, or five) other compounds for the treatment of psychiatric disorders (e.g., additional compounds for the treatment of MDD).

Preferably, the compounds of the invention may be administered in conjunction with lower doses of current treatments for psychiatric disorders, including stimulants and antidepressants. In addition, the compounds of the invention may be administered with phospholipids, e.g., lecithin, or with brain phospholipid precursors, e.g., fatty acids or lipids, or may be administered as an adjunct to standard therapy for the treatment of psychiatric disorders.

In one particular example, a creatine-containing compound is administered in combination with one or more (e.g., two, three, four, or five) of a pyrimidine, antidepressant, anticonvulsant, antianxiety, antimanic, antipsychotic, antiobsessional, sedative-hypnotic, stimulant, or anti-hypertensive medication. Non-limiting examples of pyrimidines include: cytidine monophosphate, cytidine diphosphate, cytidine triphosphate, deoxy-cytidine monophosphate, deoxy-cytidine diphosphate, and deoxy-cytidine triphosphate.

Non-limiting examples of antianxiety medications include: alprazolam, buspirone hydrochloride, chlordiazepoxide, chlordiazepoxide hydrochloride, clorazepate dipotassium, clonazepam, desipramine hydrochloride, diazepam, halazepam, hydroxyzine hydrochloride, hydroxyzine pamoate, lorazepam, meprobamate, oxazepam, prazepam, prochlorperazine maleate, prochlorperazine, prochlorperazine edisylate, and trimipramine maleate.

Non-limiting examples of anticonvulsants include: amobarbital, amobarbital sodium, paraldehyde, stiripentol, phenobarbital, phenobarbital sodium, methylphenobarbital, metharbital, barbexaclone, chlordiazepoxide, chlordiazepoxide hydrochloride, clobazam, clonazepam, clorazepate dipotassium, diazepam, divalproex sodium, midazolam, lorazepam, potassium bromide, felbamate, carbamazepine, oxcarbazepine, eslicarbazepine acetate, valproic acid, sodium valproate, divalproex sodium, vigabatrin, progabide, tiagabinc, topiramate, gabapentin, pregabalin, ethotoin, phenyloin, phenyloin sodium, mephenyloin, fosphenyloin, paramethadione, trimethadione, ethadione, beclamide, magnesium sulfate, phensuximide, primidone, brivaracetam, levetiracetam, seletracetam, ethosuximide, phesuximide, mesuximide, mephobarbital, acetazolamide, sultiame, secobarbital sodium, methazolamide, methsuximide, zonisamide, lamotrigine, pentobarbital sodium, pheneturide, phenacemide, valpromide, and valnoctamide.

Non-limiting examples of antidepressants include: isocarboxazid, moclobemide, phenelzine sulfate, selegiline, tranylcypromine sulfate, amitriptyline, amitriptyline hydrochloride, amitriptylinoxide, butriptyline, clomipramine, demexiptiline, desipramine, dibenzepin, dimetacrine, dosulepin/dothiepin, doxepin hydrochloride, imipramine, imipraminoxide, lofepramine, melitracen, metapramine, nitroxazepine, nortriptyline, noxiptiline, pipofezine, propizepine, protriptyline, phenobarbital, quinupramine, amoxapine, loxapine, maprotiline, mazindol, mianserin, mirtazapine, setiptiline, desvenlafaxine, duloxetine, milnacipram, venlafaxine hydrochloride, bupropion hydrochloride, imipramine hydrochloride, lamotrigine, trazodone hydrochloride, and trimipramine maleate, and selective serotonin reuptake inhibitors (SSRIs) including citalopram, dapoxetine, escitalopram, fluoxetine, fluvoxamine, indalpine, paroxetine, and sertraline.

Non-limiting examples of anti-manic medications include carbamazepine, divalproex sodium, gabapentin, lamotrigine, topimarate, lithium carbonate, and lithium citrate. Non-limiting examples of antiobsessional medications include fluvoxamine and clomipramine hydrochloride.

Non-limiting examples of antipsychotic medications include: acetophenazine maleate, amisulpride, aripiprazole, asenapine, cannabidiol, clopenthixol, droperidol, chlorpromazine hydrochloride, chlorprothixene, chlorprothixene hydrochloride, clozapine, fluphenazine decanoate, fluphenazine enathrate, fluphenazine hydrochloride, flupenthixol, haloperidol decanoate, haloperidol, haloperidol lactate, iloperidone, lithium carbonate, lithium citrate, levomepromazine, loxapine hydrochloride, loxapine succinate, mesoridazine besylate, molindone hydrochloride, olanzapine, paliperidone, perphenazine, periciazine, pimozide, prochlorperazine maleate, prochlorperazine, prochlorperazine edisylate, promethazine, promazine hydrochloride, quetiapine, risperidone, sertindole, thioridazine, tetrabenazine, thioridazine hydrochloride, thiothixene, thiothixene hydrochloride, triflupromazine, trifluoperzine hydrochloride, zuclopenthixol, ziprasidone, and zotepine.

Non-limiting examples of sedative-hypnotic medications include: amobarbital, amobarbital sodium, aprobarbital, butabarbital, chloral hydrate, chlordiazepoxide, chlordiazepoxide hydrochloride, clorazepate dipotassium, ethchlorvynol, flurazepam hydrochloride, glutethimide, hydroxyzine hydrochloride, pentobarbital sodium, pentobarbital, secobarbital, secobarbital sodium, phenobarbitol, phenobarbital sodium, benzodiazepines, clonazepam, diazepam, estazolam, flunitrazepam, lorazepam, methotrimeprazine hydrochloride, midazolam hydrochloride, nitrazepam, oxazepam, triazolam, temazepam, alprazolam, eszopiclone, zaleplon, zolpidem, zolpidem tartrate, zopiclone, diphenhydramine, dimenhydramine, dimenhydrinate, doxylamine, phenergan, promethazine, and quazepam.

Non-limiting examples of stimulants include caffeine, nicotine, amphetamine, dextroamphetamine sulfate, methamphetamine, methamphetamine hydrochloride, methylenedioxymethamphetamine, methylphenidate, methylphenidate hydrochloride, bupropion, atomoxetine, pemoline, reboxetine, modafinil, ampalex, CX717, carphedon, and yohimbine. Non-limiting examples of anti-hypertensive medications include clonidine, bumetanide, ethacrynic acid, furosemide, torsemide, epitizide, hydrochlorothiazide, chlorothiazide, bendroflumethiazide, indapamide, chlorthalidone, metolazone, amiloride, triamterene, spironolactone, atenolol, metoprolol, nadolol, oxprenolol, pindolol, propranolol, timolol, doxazosin, phentolamine, indoramin, phenoxybenzamine, prazosin, terazosin, tolazoline, bucindolol, carvedilol, labetalol, clonidine, methyldopa, guanfacine, amlodipine, felodipine, isradipine, lercanidipine, nicardipine, nifedipine, nimodipine, nitrendipine, diltiazem, verapamil, captopril, enalapril, fosinopril, lisinopril, perindopril, quinapril, ramipril, trandolapril, benazepril, candesartan, eprosartan, irbesartan, losartan, olmesartan, telmisartan, valsartan, eplerenone, spironolactone, sodium nitroprusside, hydralazine, clonidine, methyldopa, and moxonidine.

The one or more pyrimidines, antidepressants, anticonvulsants, antianxiety medications, antimanic medications, antipsychotic medications, antiobsessional medications, sedative-hypnotic medications, stimulants, and anti-hypertensive medications may be administered to a subject in a dose of between 0.5 mg and 1,000 mg (e.g., between 1 mg and 800 mg, 1 mg and 600 mg, 1 mg and 500 mg, 1 mg and 400 mg, 1 mg and 300 mg, 1 mg and 250 mg, 1 mg and 200 mg, 1 mg and 150 mg, 1 mg and 100 mg, 1 mg and 80 mg, 1 mg and 60 mg, 1 mg and 50 mg, 1 mg and 40 mg, 1 mg and 30 mg, 1 mg and 20 mg, 1 mg and 10 mg, and 0.5 mg and 5 mg). The one or more pyrimidines, antidepressants, anticonvulsants, antianxiety medications, antimanic medications, antipsychotic medications, antiobsessional medications, sedative-hypnotic medications, stimulants, or anti-hypertensive medications may be formulated together with one or more creatine-containing compounds (e.g., a bilayer tablet) or may be provided in a separate dosage form. The one or more pyrimidines, antidepressants, anticonvulsants, antianxiety medications, antimanic medications, antipsychotic medications, antiobsessional medications, sedative-hypnotic medications, stimulants, and anti-hypertensive medications may be administered in the same schedule (co-administered with a creatine-containing compound) or administered in an alternative schedule compared to the administration schedule of the creatine-containing compound (e.g., the creatine-containing compound may be administered two, three, or four times daily, while the one or more additional agents may be administered once a day). The one or more pyrimidines, antidepressants, anticonvulsants, antianxiety medications, antimanic medications, antipsychotic medications, antiobsessional medications, sedative-hypnotic medications, stimulants, and anti-hypertensive medications may be formulated for oral, parenteral, intravenous, subcutaneous, intramuscular, intracranial, intraorbital, ophthalmic, intraventricular, intracapsular, intraspinal, intracisternal, intraperitoneal, intranasal, or aerosol administration, or may be formulated in a sustained-release formulation.

The following examples are provided for the purpose of illustrating the invention and should not be construed as limiting.

### EXAMPLES

### Example 1

### Clinical Study of the Effect of a Creatine-Containing Compound on Male MDD Subjects

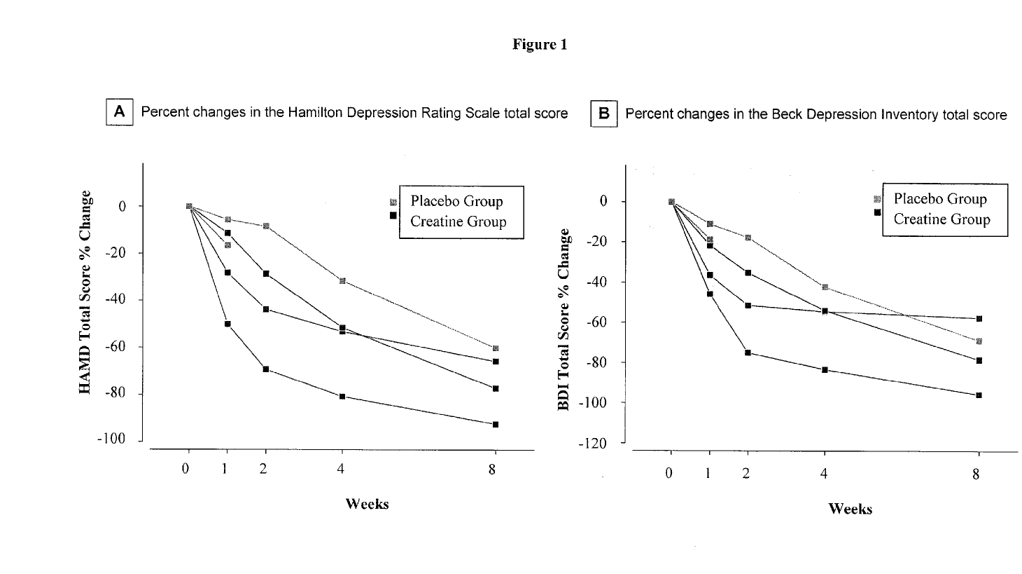

A clinical study was performed to study the effect of creatine administration on male MDD subjects. A total of 6 male subjects (mean age of 42.7 years; age range 27 to 63) were enrolled in this study. Among the subjects, 5 subjects (mean age of 38.6 years; age range of 27 to 59) underwent at least one follow-up assessment. Three subjects received 5 g/day creatine monohydrate (as a powder), and two subjects received a placebo. The MDD symptoms of the subjects were assessed using the Hamilton Depression Rating Scale (HAMD) (FIG. 1A) or the Beck Depression Inventory (BDI) (FIG. 1B). The baseline HAMD total score for intention-to-treat sample was 30.4 (standard deviation of 5.1) and the baseline BDI total score was 31 (standard deviation of 11.3). The clinical data in FIGS. 1A and 1B indicate that creatine reduces the symptoms of MDD in male subjects.

### Example 2

### Clinical Study of the Effect of a Creatine-Containing Compound on MDD Subjects Living at High Altitudes

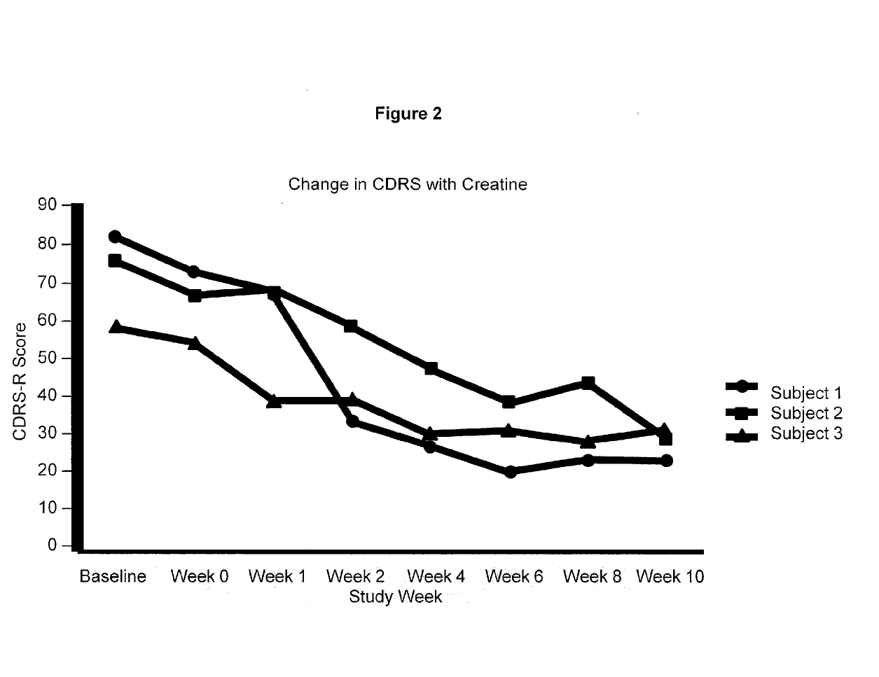

A clinical study was performed to determine the effect of creatine on adolescent MDD patients living at high altitude (Salt Lake City; elevation of 4,300 feet). In this study, three adolescents diagnosed as having MDD and living at high altitude were administered 5 g/day of creatine monohydrate (as a powder). The subjects in the study were monitored using the Children's Depression Rating Scale (CDRS). The study was performed for 1 to 10 weeks. Adolescents receiving creatine demonstrated a reduction in CDRS score (FIG. 2). These clinical data indicate that creatine reduces the symptoms of MDD in patients living at high altitude.

### Example 3

### Additional Methods for the Assessment of a Creatine-Containing Compound on Male MDD Subjects and MDD Subjects Living at High Altitude

The effect of a creatine-containing compound on male MDD subjects and on MDD subjects living at high altitude may also be assessed using phosphorus magnetic resonance spectroscopy (³¹P-MRS). Subjects having MDD have consistently shown lower levels of beta-nucleoside triphosphate (beta-NTP; primarily adenosine triphosphate in brain) and total NTP, with normal or slightly elevated high energy phosphate phosphocreatine (PCr) in the basal ganglia and frontal brain regions of MDD subjects. These findings in MDD subjects may reflect the altered turnover of ATP to PCr. Male MDD subjects and MDD subjects living at high altitudes receiving a creatine-containing compound may show improvements in these energetic abnormalities (e.g., increased PCr and reduced beta-NTP levels at baseline or an increased PCr/beta-NTP ratio).

The effect of a creatine-containing compound on MDD subjects may also be assessed using the HAMD, the BDI, the MADRAS, and the CDRS assessment protocols. A complete response to MDD treatment is considered a 50% reduction in the HAMD, the BDI, the MADRAS, or the CDRS score. Male MDD subjects and MDD subjects living at high altitudes receiving a creatine-containing compound may show improvements (e.g., at least a 20%, 30%, 40%, 50%, 60%, 70%, or 80% decrease) in HAMD, BDI, MADRAS, or CDRS score. A final HAMD score of less than or equal to 7 also indicates a complete response to MDD treatment.

While the invention has been described in connection with specific embodiments thereof it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the appended claims.

Other embodiments are within the appended claims.

1. A method of treating major depressive disorder in a male subject, comprising administering to said male a therapeutically-effective amount of a creatine-containing compound.

2. The method of claim 1, wherein the creatine-containing compound comprises a creatine salt or a creatine analog.

3. The method of claim 2, wherein said creatine analog is selected from the group consisting of cyclocreatine, homocyclocreatine, 3-guanidinopropionic acid, and 1-carboxylmethyl-2-iminohexahydropyrimidine.

4. The method of claim 1, wherein said creatine-containing compound is administered orally.

5. The method of claim 1, wherein said creatine-containing compound is administered in a dose of between 50 mg per day to 20,000 mg per day.

6. The method of claim 1, wherein said administering comprises administration of more than one dose.

7. The method of claim 6, wherein said creatine-containing compound is administered orally four times a day.

8. The method of claim 1, wherein said male is a child or an adolescent.

9. The method of claim 1, wherein said male is an adult aged 60 years or older.

10. The method of claim 1, wherein said method further comprises administering to said male a brain phospholipid or a brain phospholipid precursor selected from the group consisting of a fatty acid, glycerol, a lipid, and sphingosine.

11. The method of claim 1, wherein said method further comprises administering to said male one or more agents selected from the group consisting of an anticonvulsant, an antianxiety, an antimanic, an antipsychotic, a sedative-hypnotic, a stimulant, an anti-hypertensive medication, and a selective serotonin reuptake inhibitor.

12. A method of treating a psychiatric disorder in a subject living at a high altitude, comprising administering to said subject a therapeutically-effective amount of a creatine-containing compound.

13. The method of claim 12, wherein the creatine-containing compound comprises a creatine salt or a creatine analog.

14. The method of claim 13, wherein said creatine analog is selected from the group consisting of cyclocreatine, homocyclocreatine, 3-guanidinopropionic acid, and 1-carboxylmethyl-2-iminohexahydropyrimidine.

15. The method of claim 12, wherein said psychiatric disorder is selected from the group consisting of major depressive disorder, bipolar disorder, acute stress disorder, adjustment disorder, agoraphobia, antisocial personality disorder, anxiety disorder, avoidant personality disorder, body dysmorphic disorder, borderline personality disorder, brief psychotic disorder, conversion disorder, cyclothymic disorder, delusional disorder, dependent personality disorder, depersonalization disorder, dissociative disorder, depressive disorder, dysthymic disorder, gender identity disorder, hypochondriasis, impulse control disorder, intermittent explosive disorder, kleptomania, narcissistic personality disorder, obsessive compulsive disorder, paranoid personality disorder, posttraumatic stress disorder, psychotic disorders, schizophrenia, shared psychotic disorder, specific phobia, and attention deficit hyperactivity disorder.

16. The method of claim 15, wherein said psychiatric disorder is major depressive disorder.

17. The method of claim 12, wherein said subject lives at an elevation greater than 500 feet above sea level.

18. The method of claim 17, wherein said subject lives at an elevation greater than 1000 feet above sea level.

19. The method of claim 12, wherein said creatine-containing compound is administered orally.

20. The method of claim 12, wherein said creatine-containing compound is administered in a dose of between 50 mg per day to 2000 mg per day.

21. The method of claim 12, wherein said administering comprises administration of more than one dose.

22. The method of claim 21, wherein said creatine-containing compound is administered orally twice a day.

23. The method of claim 12, wherein said subject is a child or an adolescent.

24. The method of claim 12, wherein said subject is an adult aged 60 years or older.

25. The method of claim 12, wherein said method further comprises administering to said subject a brain phospholipid or a brain phospholipid precursor selected from the group consisting of a fatty acid, glycerol, a lipid, and sphingosine.

26. The method of claim 12, wherein said method further comprises administering to said subject one or more agents selected from the group consisting of an anticonvulsant, an antianxiety, an antimanic, an antipsychotic, an antiobsessional, a sedative-hypnotic, a stimulant, an anti-hypertensive medication, and a selective serotonin reuptake inhibitor.

27.-52. (canceled)
